(19) United States
(12) Patent Application Publication
(10) Pub. No.: US 2013/0251701 A1
(43) Pub. Date: Sep. 26, 2013

(54) ALKALINE PHOSPHATASE TO CORRECT INSULIN RESISTANCE, HYPERINSULINEMIA, AND DYSLIPIDEMIA

(71) Applicant: Zoltan Kiss, Austin, MN (US)

(72) Inventor: Zoltan Kiss, Austin, MN (US)

(73) Assignee: Zoltan Kiss Consulting, Austin, TX (US)

(21) Appl. No.: 13/879,080

(22) PCT Filed: Oct. 22, 2010
(86) PCT No.: PCT/US10/53745

§ 371 Date: Jun. 6, 2013

(51) Int. Cl. A61K 38/46; C12N 9/16

(52) U.S. Cl. CPC A61K 38/465; C12N 9/16
USPC 424/94.6; 435/196

(57) ABSTRACT

This invention provides an alkaline phosphatase or an active derivative of placental alkaline phosphatase to prevent or reduce one, two, or all three of interrelated metabolic disorders, i.e. insulin resistance, hyperinsulinemia, and dyslipidemia. Alkaline phosphatase or an active derivative of placental alkaline phosphatase is suitable to prevent or reduce diseases and disorders relating to insulin resistance, hyperinsulinemia, and dyslipidemia including type 2 diabetes, epithelial cancers, myopia, acne, acanthosis nigricans, polycystic ovary syndrome, cutaneous papillomas, hypertension, renal dysfunction, microvascular and macrovascular diseases, atherosclerosis, nonalcoholic fat liver, dyslipidemia, sleep-disordered breathing, atherosclerosis, coronary artery (heart) disease, cardiovascular disease, heart failure, accelerated aging, stroke, and neurological diseases such as Alzheimer.

### RELATED PATENTS

The present application relates to three U.S. patents, one European patent, and one allowed U.S. application as listed: (1) Alkaline phosphatase to induce weight loss or to reduce weight gain; U.S. Pat. No. 7,014,852, issued on Mar. 21, 2006; Inventor: Zoltan Kiss. (2) Placental alkaline phosphatase to control diabetes; U.S. Pat. No. 7,048,914; issued on May 23, 2006; Inventor: Zoltan Kiss. (3) Placental alkaline phosphatase to control diabetes; U.S. Pat. No. 7,501,116; issued on Mar. 10, 2009; Inventor: Zoltan Kiss. (4). Placental alkaline phosphatase to control diabetes; U.S. Pat. No. 1,572,230; Sep. 8, 2010; Inventor: Zoltan Kiss. (5) Placental alkaline phosphatase to control diabetes; application Ser. No. 12/361,310; NOA date: Sep. 8, 2010; Inventor: Zoltan Kiss. Collectively, these documents describe the positive effects of alkaline phosphatase on obesity, weight loss in type 1 diabetics, and blood glucose regulation in established type 1 and type 2 diabetic subjects. None of these documents report the effects of alkaline phosphatase on insulin resistance and hyperinsulinemia (related conditions that precede diabetes and are also associated with many physiological conditions and treatments), or dyslipidemia which contributes to atherosclerosis, coronary artery (heart) disease, cardiovascular disease, chronic heart failure and insulin resistance among other diseases.

### TECHNICAL FIELD

This invention relates to the use of alkaline phosphatase to (1) prevent or reduce insulin resistance, (2) normalize high insulin levels (hyperinsulinemia), and (3) correct dyslipidemia by reducing pathologically high levels of total cholesterol, triglyceride and free fatty acids as well as enhancing the ratio of high density lipoprotein-associated cholesterol (HDL-C) to low density lipoprotein-associated cholesterol (LDL-C).

### BACKGROUND

Insulin resistance is characterized by a reduced ability of target tissues, such as skeletal muscle, liver, and adipose tissue to respond to insulin. As a result, skeletal and adipose tissue metabolize less sugar as they normally do, while the liver produces more glucose than it normally does. The resultant hyperglycemia then leads to diabetes as well as other diseases and disorders if insulin resistance is not dealt with. Nutrient overload, even if only causing modest overweight, can induce peripheral insulin resistance [Tam, C. S., Viardot, A., Clement, K., Tordjman, J., Tonks, K., Greenfield, J. R., Campbell, L. V., Samocha-Bonet, D. and Heilbronn, L. K. (2010) Short-term overfeeding may induce peripheral insulin resistance without altering subcutaneous adipose tissue macrophages in humans. Diabetes 59: 2164-2170]. Since the year of 1960, meat consumption in the industrialized countries has increased by about 30% leading to excess nutrient supply of proteins and fat. Amino acids derived from protein degradation as well as glucose can cause insulin resistance, at least in part, via excessive activation of S6 kinase 1 followed by subsequent inhibition of insulin-stimulated glucose disposal and simultaneous down regulation of AMP-activated protein kinase in the skeletal muscle [Tremblay, F., Krebs, M., Dombrowski, L., Brehm, A., Bemmoider, E., Roth, E., Nowotny, P., Waldhausl, W., Marette, A. and Roden, M. (2005) Overactivation of S6 kinase 1 as a cause of human insulin resistance during increased amino acid availability. Diabetes 54, 2674-2684; Tremblay, F., Brüle, S., Um, S. H., Masuda, K., Roden, M., Sun, X. J., Krebs, M., Polakiewicz, R. D., Thomas, G. and Marette, A. (2007) Identification of IRS-1 Ser-1101 as a target of S6K1 in nutrient- and obesity-induced insulin resistance. Proc. Natl. Acad. Sci. USA 104, 14046-14061; Saha, A. K., Xu, X. J., Lawson, E., Deoliveira, R., Brandon, A. E., Kraegen, E. W. and Ruderman, N. B. (2010) Downregulation of AMPK accompanies leucine- and glucose-induced increases in protein synthesis and insulin resistance in rat skeletal muscle. Diabetes 59, 2436-2434].

Early insulin resistance in the skeletal muscle has the consequence that less glycogen is synthesized which promotes atherogenic dyslipidemia by channeling ingested carbohydrate into hepatic de novo lipogenesis resulting in an increase in hepatic and plasma triglyceride concentrations [Petersen, K. F., Dufour, S., Savage, D. B., Bilz, S., Solomon, G., Yonemitsu, S., Cline, G. W., Befroy, D., Zemany, L., Kahn, B. B., Papademitris, X., Rothman, D. L. and Shulman, G. I. (2007) The role of skeletal muscle insulin resistance in the pathogenesis of the metabolic syndrome. Proc. Natl. Acad. Sci. USA 104, 12587-12594, and references therein]. Increased triglyceride content in the liver is associated with insulin resistance not only in this organ but also in the skeletal muscle and adipose tissue probably mediated by very low density lipoprotein-transported triglyceride and subsequently formed free fatty acids [Kelley, D. E. and Goodpaster, B. E. (2001) Skeletal muscle trigliceride. Diabetes Care 24, 933-941; Nagle, C. A., An, J., Shiota, M., Torres, T. P., Cline, G. W., Liu, Z. X., Wang, S., Catlin, R. L., Shulman, G. I., Newgard, C. B. and Coleman, R. A. (2007) Hepatic overexpression of glycerol-sn-3-phosphate acyltransferase 1 in rats causes insulin resistance. J. Biol. Chem. 282, 14807-14815; Korenblat, K. M., Fabbrini, E., Mohammed, B. S, and Klein, S. (2008) Liver, muscle, and adipose tissue insulin action is directly related to intrahepatic trigliceride content in obese subjects. Gastroentorology 134, 1369-1375].

Fatty diet and associated obesity appears to contribute to insulin resistance by several mechanisms. There is evidence that in obesity the adipose tissue displays a pro-apoptotic phenotype. Dead adipocytes attract macrophages that then release inflammatory mediators (including tumor necrosis factor-α or TNF-α, interleukin-6 or IL-6, and interleukin-1 or IL-1) keeping the adipose tissue in an inflammatory state [Alkhouri, N., Gornicka, A., Berk, M. P., Thapaliya, S., Dixon, L. J., Kashyap, S., Schauer, P. R. and Feldstein, A. E. (2010) Adipocyte apoptosis, a link between obesity, insulin resistance and hepatic steatosis. J. Biol. Chem. 285, 3428-3438, and references therein]. Inflammation, mediated at least in part by TNF-α, results in insulin resistance in the adipose tissue and the liver accompanied by increased lipolysis and generation of long chain free fatty acids (FFA) such as palmitate [Shoelson, S. E., Lee, J. and Goldfine, A. B. (2006) Inflammation and insulin resistance. J. Clin Invest. 116, 1793-1801].

A sustained increase in plasma FFA results in a multitude of negative effects on human physiology involving multiple mechanisms. First, palmitate and similar saturated long chain FFAs induce insulin resistance via mechanisms involving reactive oxygen (ROS) and nitrogen species, the Toll-like receptor 4 (TLR-4), and downstream signaling events mediated by c-Jun NH₂ (JNK), p38 MAPK, protein kinase C, phosphatidylinositol-3-kinase, Akt kinase, insulin receptor substrate 1/2, nuclear factor-κB, and other cellular components [Silveira, L. R., Fiamoncini, J., Hirabara, S. M., Procopio, J., Cambiaghi, T. D., Pinheiro, C. H. J., Lopes, L. R. and Curi, R. (2008) Updating the effects of fatty acids on skeletal muscle. J. Cell. Physiol. 217, 1-12; Nakamura, S., Takamura, T., Nagata, N. M., Takayama, H., Misu, H., Noda, H., Nabemoto, S., Kurita, S., Ota, T., Ando, H., Miyamoto, K. I. and Kaneko, S. (2009) Palmitate induces insulin resistance in H4IIEC3 hepatocytes through reactive oxygen species produced by mitochondria. J. Biol. Chem. 284, 14809-14818; Gao, D., Nong, S., Huang, X., Lu, Y., Zhao, H., Lin, Y., Man, Y., Wang, S., Yang, J. and Li, J. (2010) The effects of palmitate on hepatic insulin resistance are mediated by NADPH oxidase 3-derived reactive oxygen species through JNK and P38 MAPK pathways. J. Biol. Chem. 285, 29965-29973].

Sustained elevation in plasma level of saturated FFAs also results in increased intramyocellular content of triglyceride and ceramide. As discussed above, triglyceride is strongly associated with insulin resistance. The case for a similar effect of ceramide is somewhat less clear, but the balance of evidence indicates that this lipid also contributes to insulin resistance in the skeletal muscle [Powell, D. J., Turban, S., Gray, A., Hajduch, E. and Hundal, H. S. (2004) Intracellular ceramide synthesis and protein kinase Cζ activation play an essential role in palmitate-induced insulin resistance in rat L6 skeletal muscle cells. Biochem. J. 382, 619-629; Adams, J. M., Pratipanawatr, T., Berria, R., Wang, E., De Fronzo, R. A., Sullards, M. C. and Mandarino, L. J. (2004) Ceramide content is increased in skeletal muscle from obese insulin-resistant humans. Diabetes 53, 25-31; Ussher, J. R., Koves, T. R., Cadete, V. J. J., Zhang, L., Jaswal, J. S., Swyrd, S. J., Lopaschuk, D. G., Proctor, S. D., Keung, W., Muoio, D. M. and Lopaschuk, G. D. (2010) Inhibition of de novo ceramide synthesis reverses diet-induced insulin resistance and enhances whole-body oxygen consumption. Diabetes 59, 2453-2464].

At pathological concentrations longer term exposure to long chain saturated FFAs have been shown to be lipotoxic to several types of cells including hepatocytes, adipocytes, endothelial cells, and islet β-cells [Kwon, G., Pappan, K. L., Marshall, C. A., Schaffer, J. E. and McDaniel, M. L. (2004) cAMP dose-dependently prevents palmitate-induced apoptosis by both protein kinase A- and cAMP-guanine nucleotide exchange factor-dependent pathways in β-cells. J. Biol. Chem. 279, 8938-8945; Jeffrey, K. O., Alejandro, E. U., Luciani, D. S., Kalynyak, T. B., Hu, X., Li, H., Lin, Y., Townsend, R. R., Polonsky, K. S, and Johnson, J. D. (2008) Carboxypeptidase E mediates palmitate-induced β-cell ER stress and apoptosis. Proc. Natl. Acad. Sci. USA 105, 8452-8457; Malhi, H., Bronk, S. F., Werneburg, N. W. and Gores, G. J. (2006) Free fatty acids induce JNK-dependent hepatocyte lipoapoptosis. J. Biol. Chem. 281, 12093-12101; Collins, J. M., Neville, M. J., Hoppa, M. B. and Frayn, K. N. (2010) De novo lipogenesis and stearoyl-CoA desaturase are coordinately regulated in the human adipocyte and protect against palmitate-induced cell injury. J. Biol. Chem. 285, 6044-6052; Staiger, K., Staiger, H., Weigert, C., Haas, C., Haring, H. U. and Kellerer, M. (2006) Saturated, but not unsaturated, fatty acids induce apoptosis of human coronary artery endothelial cells via nuclear factor-κB activation. Diabetes 55, 3121-3126]. Thus, while initially FFAs increase insulin secretion due to induction of peripheral insulin resistance, a sustained exposure of β-cells to long chain saturated FFAs also impair insulin secretion as a consequence of β-cell death [Kashyap, S., Belfort, R., Gastaldelli, A., Pratipanawatr, T., Berria, R., Pratipanawatr, W., Bajaj, M., Mandarino, L., DeFronzo, R. and Cusi, K. (2003) A sustained increase in plasma free fatty acids impairs insulin secretion in nondiabetic subjects genetically predisposed to develop type 2 diabetes. Diabetes 52, 2461-2474]. Reduced insulin secretion combined with peripheral insulin resistance leads to type 2 diabetes. Thus reduction of excess FFA level is essential for the prevention of type 2 diabetes as well as other complications due to dyslipidemia as well as associated insulin resistance and hyperinsulinemia.

While obesity and associated dyslipidemia are significant causes of insulin resistance, there are many other conditions, diseases, and treatments that also reduce the metabolic responses of skeletal muscle, liver, and adipose tissue to insulin. These include chronic exposure to insulin, hyperglycemia, pregnancy, sepsis, cancer cachexia, starvation, metabolic syndrome, acromegaly, burn trauma, breast cancer and other epithelial cancers, protease inhibitors (for the treatment of AIDS), antipsychotic drugs as well as numerous cancer and other drugs.

The most important consequence of insulin resistance is that it serves as a signal for the pancreas to secrete additional insulin to keep blood glucose levels in check; this is called compensatory hyperinsulinemia. Insulin resistance is practically always associated with hyperinsulinemia. In turn, even in healthy normoglycemic adults hyperinsulinemia is predictive of type 2 diabetes [Dankner, R., Chemit, A., Shanik, M. H., Raz, I. and Roth, J. (2009) Basal-state hyperinsulinemia in healthy normoglycemic adults is predictive of type 2 diabetes. Diabetes Care 32, 1464-1466]. This is because in many cases the pancreas eventually fails to maintain the state of compensatory hyperinsulinemia which signals the onset of impaired glucose tolerance or type 2 diabetes (i.e. when the postprandial high blood glucose level remains chronically elevated). If not treated, higher blood glucose level results in β-cell death and eventually a reduction in insulin secretion. This, combined with a further increase in insulin resistance, makes the condition of the type 2 diabetic patient worse. Due to the close causal relationship between insulin resistance, hyperinsulinemia and type 2 diabetes, a successful treatment of insulin resistance (the most important step leading to diabetes) would prevent the development of hyperinsulinemia and dyslipidemia as well as all the complications that it promotes including type 2 diabetes, epithelial (breast, prostate, colon) cancers, myopia, acne, acanthosis nigricans, polycystic ovary syndrome, cutaneous papillomas, hypertension, renal dysfunction, microvascular and macrovascular diseases, non-alcoholic fat liver, dyslipidemia, atherosclerosis, sleep-disordered breathing, coronary artery (heart) disease, cardiovascular disease, heart failure, accelerated aging, stroke, and neurological diseases such as Alzheimer [Cordain, L., Eades, M. B. and Eades, M. D. (2003) Hyperinsulinemic diseases of civilization: more than just Syndrome X. Comp. Biochem. Physiol. Part 1. 136, 95-112; Liu, H. Y., Yehuda-Shnaidman, E., Hong, T., Han, J., Pi, J., Liu, Z. and Cao, W. (2009) Prolonged exposure to insulin suppresses mitochondrial production in primary hepatocytes. J. Biol. Chem. 284, 14085-14095, and references therein].

Insulin resistance and hyperinsulinemia are causally involved in the pathogenesis of coronary artery disease and cardiovascular disease mediated by a cluster of risk factors, including dyslipidemia ((high triglyceride concentrations, an increased ratio between low density lipoprotein cholesterol (LDL-C) and high density lipoprotein cholesterol (HDL-C) as well as increased levels of circulating free saturated FFAs)), high blood pressure (hypertension), endothelial dysfunction, atherosclerosis, hypercoagulability, and obesity [Ginsberg, H. N. (2000) Insulin resistance and cardiovascular disease. J. Clin. Inv. 106, 453-458; Goff, D. C., Zaccaro, D. J., Haffner, S. M. and Saad, M. F. (2003) Insulin sensitivity and the risk of incident hypertension. Diabetes Care 26, 805-809; Reaven, G. (2005) Insulin resistance, type 2 diabetes mellitus, and cardiovascular disease. Circulation 112, 3030-3032; Laaksonen, D. E., Niskanen, L., Nyyssonen, K., Lakka, T. A., Laukkanen, J. A. and Salonen, J. T. (2008) Dyslipidaemia as a predictor of hypertension in middle-aged men. Eur. Heart J. 29, 2561-2568; Eddy, D., Schlessinger, L., Kahn, R. Peskin, B. and Schiebinger, R. (2009) Relationship of insulin resistance and related metabolic variables to coronary artery disease: A mathematical analysis. Diabetes Care 32, 361-366; Potenza, M. A., Addabbo, F. and Montagni, M. (2009) Vascular actions of insulin with implications for endothelial dysfunction. Am. J. Physiol. Endocrinol. Metab. 297, E568-E577; Forman, J. P., Choi, H. and Curhan, G. C. (2009) Uric acid and insulin sensitivity and risk of incident hypertension. Arch. Intern. Med. 169, 155-162; Lima, N. K. C., Abbasi, F., Lamendola, C. and Reaven, G. M. (2009) Prevalence of insulin resistance and related risk factors for cardiovascular disease in patients with essential hypertension. Am. J. Hypertens. 22, 106-111; Sposito, A. C. (2004) Emerging insights into hypertension and dyslipidemia synergies. Eur. Heart J. Supplements 6, (Supplement G), G8-G12].

Obesity and diabetes are also important risk factors for coronary artery disease and cardiovascular disease most probably via insulin resistance and hyperinsulinemia. However, it is important to stress that insulin resistance and hyperinsulinemia are serious risk factors for cardiovascular disease, coronary artery disease, and heart failure independent of obesity and diabetes.

A recent study indicated that in addition to increased plasma FFAs and tissue triglyceride levels, a diet rich in cholesterol can also lead to hepatic steatosis and insulin resistance [Basciano, H., Miller, A. E., Naples, M., Baker, C., Kohen, R., Xu, E., Su, Q., Allister, E. M., Wheeler, M. B. and Adeli, K. (2009) Metabolic effects of dietary cholesterol in and animal model of insulin resistance and hepatic steatosis. Am. J. Physiol. Endocrinol. Metab. 297, E462-E473]. In turn, insulin resistance promotes cholesterol synthesis and decreases cholesterol absorption [Pihlajamaki, J., Gylling, H., Miettinen, T. A. and Laakso, M. (2004) Insulin resistance is associated with increased cholesterol synthesis and decreased absorption in normoglycemic men. J. Lipid Res. 45, 507-512; Gylling, H., Hallikainen, M., Pihlajamaki, J., Simonen, P., Kuusisto, J., Laakso, M. and Miettinen, T. A. (2010) Insulin sensitivity regulates cholesterol metabolism to a greater extent than obesity: lessons from the METSIM study. J. Lipid Res. 51, 2422-2427]. Increasing levels of cholesterol also directly inhibit glucose stimulated insulin secretion [Hao, M., Head, W. S., Gunawardana, S. C., Hasty, A. H. and Piston, D. W. (2007) Direct effect of cholesterol on insulin secretion: A novel mechanism for pancreatic β-cell dysfunction. Diabetes 56, 2328-2338]. Thus, the ingestion of excess fat including cholesterol starts the process of insulin resistance which then results in increased synthesis of cholesterol that then further increases, along with increased triglyceride and FFAs, insulin resistance and compensatory hyperinsulinemia. Eventually, high cholesterol reduces insulin secretion which, together with insulin resistance, leads to persistent hyperglycemia, i.e. diabetes. Because of the close causal interrelationships among insulin resistance, dyslipidemia and hyperinsulinemia [Shanik, M. H., Xu, Y., Skrha, J., Dankner, R., Zick, Y. and Roth, J. (2008) Insulin resistance and hyperinsulinemia: Is hyperinsulinemia the cart or the horse? Diabetes Care 31 (Suppl. 2) S262-S268], treatment of any of these conditions will improve the other two conditions as well. Combined early reduction of dyslipidemia, insulin resistance and hyperinsulinemia then should act to delay or prevent diabetes and other related conditions such as epithelial (breast, prostate, colon) cancers, myopia, acne, acanthosis nigricans, polycystic ovary syndrome, cutaneous papillomas, hypertension, renal dysfunction, microvascular and macrovascular diseases, atherosclerosis, nonalcoholic fat liver, dyslipidemia, sleep-disordered breathing, coronary artery (heart) disease, cardiovascular disease, heart failure, accelerated aging, stroke, and neurological disorders such as Alzheimer.

Recently, most scientists working on atherosclerotic cardiac disease and chronic heart failure reached a consensus stating that low HDL-C (below 40 mg/dL) is a risk factor for these diseases and that significant elevation of HDL-C is as important as lowering LDL-C to prevent and combat this disease [Chapman, J. (2005) Beyond LDLcholesterol reduction: the way ahead of managing dyslipidemia. Eur. Heart J. Supplements, 7 (Supplement F), F56-F62]. An ideal agent would significantly reduce the ratio between LDL-C and HLD-C in addition to lowering triglyceride and FFAs. Today, the best, although still not ideal, agents for that purpose are the inhibitors of 3-hydroxy-3-methylglutaryl coenzyme A reductase (statins), such as Atorvastatin (Lipitor), Pravastatin (Pravachol), Lovastatin (Mevacor), Simvastatin (Zocor), and Fluvastatin (Lescol). Statins seem to be particularly effective when used together with niacin (nicotinic acid or vitamin B₃) which is mostly effective in raising HDL-C levels while statins primarily reduce LDL-C [van der Harst, P., Voors, A. A., van Gilst, W. H., Bohm, M. and van Veldhuisen, D. J. (2006) Statins in the treatment of chronic heart failure: Biological and clinical considerations. Cardiovascular Res. 71, 443-454; Shepherd, J. (2005) Raising HDLcholesterol and lowering CHD risk: does intervention work? Eur. Heart J. Supplements 7 Supplement F), F15-F22].

Physical exercise and reduction of consumption of fat-rich and high-glycemic index diets help to control insulin resistance and hyperinsulinemia, however, presently there are no FDA approved drugs available to treat these conditions. Clinical studies indicated that metformin might be useful for the prevention of insulin resistance and hyperinsulinemia, but its approval for these purposes remains to be granted, although widely anticipated.

Insulin resistance may also develop due to high-glycemic index carbohydrate diet. Such diet can cause longer periods of hyperinsulinemia and increased postprandial level of serum FFA. These two conditions together cause insulin resistance and a pre-diabetic state that, if sustained, can develop into diabetes [Ludwig, D. S. (2002) The glycemic index: Physiological mechanisms relating to obesity, diabetes, and cardiovascular disease. JAMA 287, 2414-2423]. An agent that increases insulin sensitivity of target tissues would be expected to reduce carbohydrate-induced hyperinsulinemia. The reverse is also true; i.e. an agent that reduces or prevents hyperinsulinemia due to increased and prolonged insulin secretion triggered by carbohydrate diet would reduce or prevent insulin resistance (due to chronic exposure of target tissues to insulin) and the increase in postprandial serum level of FFAs. In both scenarios, the agent would prevent or reduce the development of diabetes and related complications.

Many older subjects develop insulin resistance due to physical inactivity and/or obesity [Amati, F., Dube, J. J., Coen, P. M., Stefanovic-Racic, M., Toledo, F. G. S, and Goodpaster, B. H. (2009) Physical inactivity and obesity underlie the insulin resistance of aging. Diabetes Care 32, 1547-1549]. Insulin resistance may then lead to many disorders and diseases (epithelial cancers, myopia, acne, acanthosis nigricans, polycystic ovary syndrome, cutaneous papillomas, hypertension, renal dysfunction, microvascular and macrovascular diseases, atherosclerosis, nonalcoholic fat liver, dyslipidemia, sleep-disordered breathing, coronary artery (heart) disease, cardiovascular disease, heart failure, accelerated aging, stroke, and neurological disorders such as Alzheimer) including type 2 diabetes and associated complications. While in younger age groups increased physical activity can reduce insulin resistance, for old people, often struck with serious immobility, strenuous exercise is usually not an option. However, a drug which preventively or curatively can deal with insulin resistance would be expected to reduce a range of diseases, as listed above, that old people may be afflicted with due to immobility and associated insulin resistance.

In summary, there are multiple etiological factors that may be involved in the development of insulin resistance and associated hyperinsulinemia and dyslipidemia. In addition to heritable genetic factors, overeating food rich in fat and/or sugar, and/or protein, physical inactivity, overweight, obesity, many diseases and disorders or disease treatments (chronic exposure to insulin, hyperglycemia, pregnancy, sepsis, cancer cachexia, starvation, metabolic syndrome, acromegaly, burn trauma, breast cancer and other epithelial cancers, protease inhibitors for the treatment of AIDS, antipsychotic drugs as well as numerous cancer and other drugs) can play key roles. The triage of insulin resistance, hyperinsulinemia and dyslipidemia frequently leads to type 2 diabetes and many other diseases and disorders as already listed above. Reduction or prevention of this triage of metabolic abnormalities should delay or prevent development of type 2 diabetes as well as other diseases and disorders like epithelial cancers, myopia, acne, acanthosis nigricans, polycystic ovary syndrome, cutaneous papillomas, hypertension, renal dysorder, microvascular and macrovascular diseases, atherosclerosis, nonalcoholic fat liver, dyslipidemia, sleep-disordered breathing, coronary artery (heart) disease, cardiovascular disease, heart failure, accelerated aging, stroke, and neurological diseases such as Alzheimer.

### SUMMARY OF THE INVENTION

The present invention relates to the use of an alkaline phosphatase or an active derivative of placental alkaline phosphatase (PLAP) for reducing a triage of interrelated metabolic disorders including insulin resistance, hyperinsulinemia, and dyslipidemia, and thereby reducing or preventing the occurrence of type 2 diabetes as well as other diseases and disorders including epithelial cancers, myopia, acne, acanthosis nigricans, polycystic ovary syndrome, cutaneous papillomas, hypertension, renal dysfunction, microvascular and macrovascular diseases, atherosclerosis, nonalcoholic fat liver, dyslipidemia, sleep-disordered breathing, atherosclerosis, coronary artery (heart) disease, cardiovascular disease, heart failure, accelerated aging, stroke, and neurological diseases such as Alzheimer.

In one embodiment, subjects who are at risk of developing insulin resistance or who have already developed insulin resistance due to, for example, a genetic abnormality, a diet rich in fat and/or sugar/and or protein, overweight, obesity, chronic exposure to insulin, hyperglycemia, pregnancy, sepsis, cancer cachexia, starvation, metabolic syndrome, acromegaly, burn trauma, cancer as well as treatments with protease inhibitors (for the treatment of AIDS), antipsychotic drugs, and cancer drugs, are regularly treated with alkaline phosphatase or an active derivative of PLAP to prevent or treat this condition and reduce or prevent associated hyperinsulinemia and dyslipidemia.

In another embodiment, subjects who are at risk of developing or already developed hyperinsulinemia are regularly treated with alkaline phosphatase or an active derivative of PLAP to prevent or treat this condition and reduce or prevent associated insulin resistance and dyslipidemia.

In a further embodiment, subjects who are at risk of developing or already developed dyslipidemia are regularly treated with alkaline phosphatase or an active derivative of PLAP to prevent or treat this condition and reduce or prevent associated insulin resistance and dyslipidemia.

In additional embodiments an alkaline phosphatase or an active derivative of PLAP is used to simultaneously prevent or treat insulin resistance, hyperinsulinemia, and dyslipidemia to reduce the risk of developing type 2 diabetes or other related diseases and disorders like epithelial cancers, myopia, acne, acanthosis nigricans, polycystic ovary syndrome, cutaneous papillomas, hypertension, renal dysfunction, microvascular and macrovascular diseases, nonalcoholic fat liver, dyslipidemia, sleep-disordered breathing, atherosclerosis, coronary artery (heart) disease, cardiovascular disease, heart failure, accelerated aging, stroke and neurological diseases such as Alzheimer.

In yet another embodiment, subjects with type 2 diabetes whose hyperglycemia but not dyslipidemia is well controlled by other drugs are regularly treated with an alkaline phosphatase or an active derivative of PLAP to reduce dyslipidemia and thereby the risk of atherosclerosis, coronary artery (heart) disease, cardiovascular diseases and heart failure.

In this invention the terms for insulin resistance, hyperinsulinemia, and dyslipidemia are defined by the relevant science literature. Accordingly, insulin resistance means an impaired ability of insulin to regulate carbohydrate, lipid, and protein/amino acid metabolism in key target tissues such as the skeletal muscle, liver, and adipose tissue. Insulin resistance can also develop in other tissues, such as the heart. Hyperinsulinemia means the pancreatic islets almost invariably release insulin above the normal level to meet the metabolic demands of peripheral tissues in response to insulin resistance. Dyslipidemia means atherogenic lipid abnormalities including hypertriglyceridemia, decreased HDL-C and relative (compared to HDL-C) or absolute increase in LDL-C as well as increased plasma level of saturated long chain FFAs, especially palmitate. Recently, increased synthesis of ceramide also is considered to be part of the dyslipidemia condition.

### DETAILED DESCRIPTION OF THE INVENTION

### The Active Component

Alkaline phosphatases (E.C.3.1.3.1) are dimeric enzymes that catalyze the hydrolysis of phosphomonoesters with release of inorganic phosphate and alcohol [Millan, J. L. (2006) Alkaline phosphatases: Structure, substrate specificity and functional relatedness to other members of a large superfamily of enzymes. Purinergic Signalling 2, 335-341; Kozlenkov, A., Manes, T., Hoylaerts, M. F. and Millan, J. L. (2002) Function assignment to conserved residues in mammalian alkaline phosphatases. J. Biol. Chem. 277, 22992-22999]. In humans, three of the four isozymes, including the intestinal (IAP), placental (PLAP), and germ cell (GCAP) enzymes, are tissue-specific and exhibit 90-98% homology. The fourth isozyme, tissue-nonspecific alkaline phosphatase (TNAP), is expressed in the bone, liver and kidney and is about 50% identical with the other three alkaline phosphatases with maintaining a remarkable sequence homology at the catalytic site. Various alkaline phosphatases are expressed from bacteria to humans with the main features of enzyme's properties being conserved [Millan, J. L. (2006) Alkaline phosphatases: Structure, substrate specificity and functional relatedness to other members of a large superfamily of enzymes. Purinergic Signalling 2, 335-341]. For example, both bacterial and mammalian alkaline phosphatases are non-specific phosphatases commonly removing phosphate groups from proteins, nucleotides, alkaloids, and lipid phosphates such as phosphatidic acid and phospholipid precursors including phosphoethanolamine and phosphocholine [Millan, J. L. (2006) Alkaline phosphatases: Structure, substrate specificity and functional relatedness to other members of a large superfamily of enzymes. Purinergic Signalling 2, 335-341; Huang, K. P., Robinson, J. C. and Chou, J. Y. (1976) Phosphoprotein-phosphatase activity associated with human placental alkaline phosphatase. Biochem. Biophys. Res. Comm. 70, 186-192; Swamp, G., Cohen, S, and Garbers, D. L. (1981) Selective dephosphorylation of proteins containing phosphotyrosine by alkaline phosphatase. J. Biol. Chem. 256, 8197-8201; Ekman, P. (1991) Dephosphorylation with alkaline phosphatase of histone and fibrinogen phosphorylated with protein kinase C in vitro. Upsala J. Med. Sci. 96, 95-102; Sumikawa, K., Saeki, K., Okochi, T., Adachi, and Nishimura, H. (1987) Hydrolysis of phosphatidate by human placental alkaline phosphatase. Clin. Chim. Acta 167, 321-328]. These actions of alkaline phosphatases may result in molecules with specific biological functions. For example, hydrolysis of the phosphate ester of phosphatidic acid yields 1,2-diacylglycerol, a potent activator of specific protein kinase C isozymes that in turn regulate many cellular processes including the actions of insulin. Also, sequential hydrolysis of ATP, ADP and 5′-AMP yields adenosine that downregulates inflammation via A_{2B} or A_{2A} receptors [Ohta, A. and Sitkovsky, M. (2001) Role of G-protein-coupled adenosine receptors in downregulation of inflammation and protection from tissue damage. Nature 414, 916-920; Reece, T. B., Ellman, P. I., Maxey, T. S., Crosby, I. K., Warren, P. S., Chong, T. W., LeGallo, R. D., Linden, J., Kern, J. A., Tribble, C. G. and Kron, I. L. (2005) Adenosine_{2A} receptor activation reduces inflammation and preserves pulmonary function in an in vivo model of lung transplantation. J. Thorac. Cardiovasc. Surg. 129, 1137-1143], promotes cell survival in serum free medium [Huang, N. K., Lin, Y. W., Huang, C. L., Messing, R. O. and Che™, Y. (2001) Activation of protein kinase A and atypical protein kinase C by A_{2A} adenosine receptor antagonizes apoptosis due to serum deprivation in PC12 cells. J. Biol. Chem. 276, 13838-13846; She, Q. B., Mukherjee, J. J., Chung, T. and Kiss, Z. (2000) Placental alkaline phosphatase, insulin, and adenine nucleotides or adenosine synergistically promote long-term survival of serum-starved mouse embryo and human fetus fibroblasts. Cellular Signaling 12, 659-665], exerts cardioprotective effects via phospholipases C and D [Parsons, M., Young, L., Lee, J. E., Jacobson, K. A. and Liang, B. T. (2000) Distinct cardioprotective effects of adenosine mediated by differential coupling of receptor subtypes to phospholipases C and D. FASEB J. 14, 1323-1431], and possess antilipolytic activity [Dhalla, A. K., Santikul, M., Smith, M., Wong, M. Y., Shryock, J. C. and Belardinelli, L. (2007) Antilipolytic activity of a novel partial A₁ adenosine receptor agonist devoid of cardiovascular effects: Comparison with nicotinic acid. J. Pharmacol. Exp. Ther. 321, 327-333]. Although none of the products of the alkaline phosphatase activity, including diacylglycerol and adenosine, have been implicated in the normalization of insulin resistance, hyperinsulinemia, and dyslipidemia, the above examples strongly indicate that once one of the alkaline phosphatases is shown to correct these deficiencies, its effects will be shared, at least in part, by other alkaline phosphatases. Thus, in the context of wide ranging common effects of various alkaline phosphatases, the ability of PLAP to correct the deficiencies of insulin resistance, hyperinsulinemia, and dyslipidemia, as demonstrated here, is shared at least in part by other alkaline phosphatases.

Not all biological effects of alkaline phosphatases may involve the phosphatase activity. For example, it has been demonstrated that both PLAP and IALP can serve in the intestine as receptors for the bacterial product hemolysin with no indication for a role of the catalytic activity [Wada, A., Wang, A. P., Isomoto, H., Satomi, Y., Takao, T., Takahashi, A., Awata, S., Nomura, T., Fujii, Y., Kohno, S., Okamoto, K., Moss, J., Millan, J. L. and Hirayama, T. (2005) Placental and intestinal alkaline phosphatases are receptors for Aeromonas sobria hemolysin. Internat. J. Med. Microbiol. 294, 427-435]. Similarly, although all alkaline phosphatases are glycoproteins, the receptor function of PLAP did not require N-linked glycosylation of the protein [Wada, A., Wang, A. P., Isomoto, H., Satomi, Y., Takao, T., Takahashi, A., Awata, S., Nomura, T., Fujii, Y., Kohno, S., Okamoto, K., Moss, J., Millan, J. L. and Hirayama, T. (2005) Placental and intestinal alkaline phosphatases are receptors for Aeromonas sobria hemolysin. Internat. J. Med. Microbiol. 294, 427-435]. These observations increase the likelihood that the effects of alkaline phosphatases on insulin resistance, hyperinsulinemia, and dyslipidemia may not require the alkaline phosphatase activity and/or glycosylation of these proteins.

Previous work showed that PLAP generally enhances the proliferation and survival of normal cells such mouse embryo fibroblasts, fibroblast-like cells derived from the lung of human fetus, adult human fibroblasts, epidermal cells, stem cells as well as proliferating precursors of myotubes and adipocytes [She, Q.-B., Mukherjee, J. J., Huang, J.-S., Crilly, K. S, and Kiss, Z. (2000) Growth factor-like effects of placental alkaline phosphatase in human fetus and mouse embryo fibroblasts. FEBS Letters, 468, 163-167; She, Q.-B., Mukherjee, J. J., Chung, T. and Kiss, Z. (2000) Placental alkaline phosphatase, insulin, and adenine nucleotides or adenosine synergistically promote long-term survival of serum-starved mouse embryo and human fetus fibroblasts. Cellular Signaling 12, 659-665; Z. Kiss, Combinations of human proteins to enhance in vivo viability of stem cells and progenitor cells; U.S. Pat. No. 7,786,082; issued on: Aug. 31, 2010. Inventor: Zoltan Kiss; Use of placental alkaline phosphatase to promote skin cell proliferation; U.S. Pat. No. 7,374,754, issued on May 20, 2008; Inventor: Zoltan Kiss]. In contrast, PLAP was shown to reduce the viability of cancer cells in vitro and in vivo [Alkaline phosphatase to reduce skin cancer. U.S. Pat. No. 7,718,170 issued on May 18, 2010, Inventor: Zoltan Kiss; Use of alkaline phosphatase to maintain healthy tissue mass in mammals, U.S. Pat. No. 7,695,714 issued on Apr. 13, 2010; Inventor, Zoltan Kiss]. These selective effects of PLAP on cell proliferation and survival are most likely unrelated or only partially related to its effects on insulin sensitivity, insulin level, and lipid metabolism. This feature of PLAP is similar to some of the other growth factors, such as insulin and insulin-like growth factor 1 that are well known to affect cell proliferation/survival and cell metabolism by essentially separate cellular mechanisms.

For the reason that different alkaline phosphatases share many common effects with differences only in the magnitude of effects, the present invention, that uses PLAP as the principal alkaline phosphatase, teaches that the observed effects of this protein on insulin resistance, hyperinsulinemia, and dyslipidemia may also be elicited, at least in part, by other alkaline phosphatases. For simplicity, in the specification the terms “PLAP” and “alkaline phosphatase (AP)” are used interchangeably.

It may be possible to synthesize or develop an active derivative that is a smaller fragment of PLAP's amino acid sequence and/or mutated to diminish its catalytic activity and still demonstrates efficacy similar to that of native protein. In this context, it has been demonstrated that the alkaline phosphatase activity of PLAP is not required to stimulate mitogenesis. For example, both digestion of PLAP with the protease bromelain and elimination of alkaline phosphatase activity through mutation provided active derivatives with respect to cell proliferation [Use of placental alkaline phosphatase to promote skin cell proliferation; U.S. Pat. No. 7,374,754, issued on May 20, 2008; Inventor, Zoltan Kiss]. In another example, a secreted form of PLAP was expressed in mammalian cells in which the glycosylphosphatidylinositol anchoring sequence (29 amino acids) was replaced with a shorter FLAG octapeptide [Kozlenkov, A., Manes, T., Hoylaerts, M. F. and Millan, J. L. (2002) Function assignment to conserved residues in mammalian alkaline phosphatases. J. Biol. Chem. 277, 22992-22999]; this shortened PLAP stimulated mitogenesis in mouse and human fibroblasts [Use of placental alkaline phosphatase to promote skin cell proliferation; U.S. Pat. No. 7,374,754, issued on May 20, 2008; Inventor, Zoltan Kiss]. Other site specific mutations can be introduced into PLAP (and certainly into other alkaline phosphatases as well) that do not alter the catalytic activity but changes its membrane binding [Lowe, M. E. (1992) Site-specific mutations in the COOH-terminus of placental alkaline phosphatase: a single amino acid change converts a phosphatidylinositol-glycan-anchored protein to a secreted protein. J. Cell Biol. 116, 799-807]. These examples show that shortened, altered, and mutated forms of PLAP can be generated that retain enzyme and biological activity. From the perspective of the present invention “active AP” or an “active derivative of PLAP” means that the protein detectably reduces each of the following disorders; insulin resistance, hyperinsulinemia, and dyslipidemia. An active derivative of PLAP can be made by genetic modification of the native protein usually resulting in a recombinant product. An active derivative of PLAP can also be synthesized chemically, or it may be produced from PLAP by a proteolytic process. Concerning chemical synthesis, solid-phase synthesis techniques may be used to obtain PLAP or an active AP derivative.

Recombinant methods of obtaining appropriate preparations of PLAP or active derivatives are feasible. Using the cDNA of PLAP, recombinant protein may be produced by one of the many known methods for recombinant protein expression. PLAP has been cloned and has already been expressed in different cell types [Kozlenkov, A., Manes, T., Hoylaerts, M. F. and Millan, J. L. (2002) Function assignment to conserved residues in mammalian alkaline phosphatases. J. Biol. Chem. 277, 22992-22999; Henthorn, P., Zervos, P., Raducha, M., Harris, H. and Kadesh, T. (1988) Expression of a human placental alkaline phosphatase gene in transfected cells: Use as a reporter for studies of gene expression. Proc. Natl. Acad. Sci. USA 85, 6342-6346; Chen, Y. H., Chang, T. C. and Chang, G. G. (2004) Functional expression, purification, and characterization of the extra stable human placental alkaline phosphatase in the Pichia pastoris system. Protein Expression & Purification 36, 90-99; Becerra-Artega, A., Mason, H. S. and Shuler, M. I. (2006) Production, secretion, and stability of human secreted alkaline phosphatase in tobacco NT1 cell suspension cultures. Biotechnol. Prog. 22, 1643-1649]. Potentially, recombinant PLAP and other alkaline phosphatases as well as an active derivative of PLAP may also be expressed in and obtain from, for example, cow's milk, goat's milk, and plant (for example, barley, rice, corn, tobacco) seeds or leaves.

A preparation of human PLAP may also be obtained by extraction from placental tissue. Human placenta synthesizes the enzyme during pregnancy so that toward the end of the third term, the level of PLAP in the placenta tissue and the maternal and fetal blood becomes very high. Therefore, a preparation of PLAP may be obtained by butanol extraction of homogenized placenta. Other methods of extraction from placental tissue are also suitable. Other alkaline phosphatases may be extracted and purified from blood, liver, and other tissues.

It may be suitable to purify the raw placental extracts of PLAP before using it in the method of the present invention. Raw placental extracts may contain other proteins, lipids, proteolipids, carbohydrates, metals, vitamins, and the like that may cause unexpected side effects when administered to a patient. Therefore, it may be suitable to use a known purification method to remove these contaminants from the raw placental extract.

If the recombinant alkaline phosphatase or an active derivative of PLAP is derived from plants that are used for human consumption without restriction, after careful testing a protein extract from such source may be used for oral consumption without further purification of the protein. Any of the available suitable extraction methods known in the food industry can be used for the production of protein extracts from plants.

Source of PLAP Used for this Invention:

Highly purified PLAP was prepared from a partially purified commercial human PLAP preparation (Sigma, St Louis, Mo.) as described earlier in detail including presentation of a gel picture demonstrating the presence of a single PLAP band in the final preparation [She, Q.-B., Mukherjee, J. J., Huang, J.-S., Crilly, K. S, and Kiss, Z. (2000) Growth factor-like effects of placental alkaline phosphatase in human fetus and mouse embryo fibroblasts. FEBS Letters, 468, 163-167; Placental alkaline phosphatase to control diabetes; U.S. Pat. No. 7,501,116; issued on Mar. 10, 2009; Inventor: Zoltan Kiss].

Patient Groups Treated with PLAP or Another AP in this Invention:

A human or another mammal with any condition that predisposes to or already led to insulin resistance, and/or hyperinsulinemia, and/or dyslipidemia may be treated with alkaline phosphatase or an active derivative of PLAP. For example, such condition may be a genetic abnormality, a diet rich in fat and/or sugar, and/or protein, overweight, obesity, chronic exposure to insulin (including regular treatment with insulin), hyperglycemia, pregnancy, sepsis, cancer cachexia, starvation, metabolic syndrome, acromegaly, burn trauma, cancer as well as treatment with a protease inhibitor (for the treatment of AIDS), an antipsychotic drug, or a cancer drug. The purpose of such treatment is to prevent or reduce diseases and disorders that are related to insulin resistance, hyperinsulinemia and dydlipidemia including epithelial cancers, myopia, acne, acanthosis nigricans, polycystic ovary syndrome, cutaneous papillomas, hypertension, renal dysfunction, microvascular and macrovascular diseases, atherosclerosis, nonalcoholic fat liver, dyslipidemia, sleep-disordered breathing, atherosclerosis, coronary artery (heart) disease, cardiovascular disease, heart failure, accelerated aging, stroke, and neurological diseases such as Alzheimer. Patients qualified for PLAP treatment may have already been afflicted with one or more of the above listed diseases or disorders and might receive corresponding treatments other than alkaline phosphatase. Another purpose of the treatment is to prevent escalation of the insulin resistance, hyperinsulinemia and dydlipidemia disorders into type 2 diabetes. In this invention, it is not the goal to use alkaline phosphatase for the treatment of obesity, type 2 diabetes, or type 1 diabetes.

### Methods of Treatments

PLAP or another AP (or an active AP derivative) may be administered by a variety of methods for the prevention or reduction of insulin resistance, hyperinsulinemia, and dydlipidemia.

In one embodiment, an alkaline phosphatase solution is administered to the human or another mammal by injection. Any suitable method of injection, such as intravenous, intraarterial, intraportal, intramuscular, intraperitoneal, intradermal, infusion or subcutaneous may be used. A device, for example an osmotic minipump inserted under the skin or elsewhere in the body, may be used for providing controlled release of alkaline phosphatase. For injection of a solution of PLAP or another alkaline phosphatase, the protein may be dispersed in any physiologically acceptable carrier that does not cause an undesirable physiological effect. Examples of suitable carriers include physiological saline and phosphate-buffered saline. The injectable solution may be prepared by dissolving or dispersing a suitable preparation of the active protein component in the carrier using conventional methods. As an example only, a suitable composition for the practice in the methods includes PLAP in a 0.9% physiological salt solution to yield a total protein concentration of 10 mg/ml. Another suitable solution includes PLAP in a 0.9% physiological salt solution to yield a total protein concentration of 20 mg/ml. A third suitable solution includes PLAP in a 0.9% physiological salt solution to yield a total protein concentration of 50 mg/ml. Alternatively, PLAP may be enclosed in liposomes such as immunoliposomes, or other delivery systems or formulations that are known in the art.

A suitable dosage for systemic administration of alkaline phosphatase to prevent or treat insulin resistance, hyperinsulinemia, and dyslipidemia may be calculated in grams of the active agent(s) per square meter of body surface area for the subject. A common way to express a suitable dosage is grams of active agent per square meter of body surface area for the subject. Several formulas are known for estimating a human subject's body surface area, based on the human's height (in cm) and mass (in kg). Table 1 lists a variety of known formulas for estimating body surface area (BSA) proposed by researchers. Other suitable formulas may likewise be employed.

TABLE 1
Formulas for estimating body surface area.

| Author(s) | BSA formula |
|---|---|
| Du Bois and | BSA (m²) = Mass(kg)^{0.425} × Height(cm)^{0.725} × 0.007184 |
| Du Bois | |
| Gehan and | BSA (m²) = Mass(kg)^{0.51456} × |
| George | Height(cm)^{0.42246} × 0.02350 |
| Haycock | BSA (m²) = Mass(kg)^{0.5378} × Height(cm)^{0.3964} × 0.024265 |
| Mosteller | BSA (m²) = Mass(kg)^{0.5} × Height(cm)^{0.5} × 0.016666 |

In one embodiment, the therapeutically effective amount of PLAP is between about 0.01 to 2.5 g per m² body surface of the mammal. In another embodiment, the therapeutically effective amount of PLAP is between 0.1 to 1 g per m² body surface of the mammal.

In one embodiment, the therapeutically effective amount of alkaline phosphatase may be administered once or twice daily. In another embodiment, the dose is administered twice or three times weekly. In still another embodiment, administration is performed once a week. The half life time for different alkaline phosphatases is different which needs to be taken into account when the frequency of the treatment is decided. For example, the biological half-life time of PLAP is relatively long, about 7 days [Clubb, J. S., Neale, F. C. and Posen, S. (1965) The behaviour of infused human placental alkaline phosphatase in human subjects. J. Lab. & Clin. Med. 66, 493-507]; thus, for this alkaline phosphatase one suitable application is twice a week or once a week. In contrast, the biological half life time for IALP is only about 40 minutes [Clubb, J. S., Neale, F. C. and Posen, S. (1965) The behaviour of infused human placental alkaline phosphatase in human subjects. J. Lab. & Clin. Med. 66, 493-507]; thus, for this alkaline phosphatase a suitable application is twice or once a day. Another factor to consider when determining the effective amount is whether the alkaline phosphatase is used alone or used as part of a more complex regimen involving other treatments. Thus, if a patient is simultaneously or alternatively treated with both PLAP and another therapy, the effective tolerated amount of the former may be less compared to a regimen when the subject is treated with PLAP alone. For example, if PLAP used together with a statin both lowering LDL-C and triglyceride, then a lower dose of PLAP may be sufficient for obtaining the same result.

At the present, based on the excellent toxicological profile of PLAP and other alkaline phosphatases in humans [Clubb, J. S., Neale, F. C. and Posen, S. (1965) The behaviour of infused human placental alkaline phosphatase in human subjects. J. Lab. & Clin. Med. 66, 493-507], there is reason to believe that these proteins may be used in combination with any other drug used to treat any disease or disorder. For example, alkaline phosphatase may be used together with statins (Atorvastatin or Lipitor, Pravastatin or Pravachol, Lovastatin or Mevacor, Simvastatin or Zocor, and Fluvastatin or Lescol) with or without niacin for a better result to reduce LDL-C, enhance HDL-C as well as reduce triglyceride and FFAs.

PLAP and other alkaline phosphatases may also be taken orally in the form of a liquid, tablet, gel, capsule, gelcapsule or the like. As it will be demonstrated under the Examples, mice that had free access to high fat diet (HFD) and PLAP-free water had higher levels of fatty acids in the serum than mice that had free access to both HFD and PLAP containing water. This result is interpreted to mean that PLAP, and by implication other alkaline phosphatases as well, can be used orally to reduce dyslipidemia. Since saturated long chain free fatty acids (FFAs), like palmitate (also known as palmitic acid), play roles in insulin resistance, hyperinsulinemia and dyslipidemia, orally taken alkaline phosphatase or an active derivative of PLAP should be able to improve the severity of these disorders and reduce the already listed health consequences of these disorders. In one embodiment of the invention, on one occasion the subject weighing 100 kg drinks about 2 dL of a liquid containing 1 mg to 500 mg PLAP or another alkaline phosphatase (or an active derivative of PLAP). In another embodiment of the invention, on one occasion the subject weighing 100 kg drinks about 2 dL of a liquid containing 20 mg to 200 mg PLAP or another alkaline phosphatase (or an active derivative of PLAP). The alkaline phosphatase may be dissolved in water, milk, any non-alcoholic juice, or alcoholic drinks containing only moderate amount (up to 15%) of alcohol that does not precipitate the protein. The alkaline phosphatase containing solution may contain any commercially available and used additive or additives to increase the taste or efficacy of the protein. For example, such additive(s) can be sugars, vitamins, essential elements, calcium, magnesium, zinc, and plant or fruit extracts. The criterion for such additive is that it does not reduce the biological actions of alkaline phosphatase. The volume of the alkaline phosphatase containing solution prepared for one drink may be lower or higher than 2 dL; in this case the amount of dissolved alkaline phosphatase needs to be correspondingly adjusted.

In other embodiments of the invention the alkaline phosphatase is delivered orally in the form of a liquid, tablet, gel, capsule, gel capsule, or a semisolid mixture containing other food components. In each case the alkaline phosphatase is mixed with one or more carriers selected to best suit the goal of treatment using methods practiced in the pharmaceutical industry. In addition to the alkaline phosphatase (1 mg to 500 mg or 20 mg to 200 mg for one application per 100 kg body weight) and the carrier, the tablet, gel, capsule, and gel capsule may contain any component that is presently used in the pharmaceutical field to ensure firmness, stability, solubility and appropriate taste. The alkaline phosphatase (1 mg to 500 mg or 20 mg to 200 mg for one application per 100 kg body weight) can also be mixed with certain semisolid food items; for example it can be mixed with a vegetable dish or mashed potato. Any additional component added to the liquid, tablet, gel, capsule, and gel capsule or the semisolid food items will not reduce the biological actions of alkaline phosphatase.

If the recombinant alkaline is derived from plant seeds or plant leaves that are normally can be used for human consumption without restriction, a protein extract from such source may be used for oral consumption without further purification of the protein. Appropriate safety guidelines and approval procedures will have to be observed for the oral use of such protein extract. The amount of extract to be consumed will depend on the rate of expression of the alkaline phosphatase. For example, if PLAP is expressed in barley seed and it represents about 10% of the total protein, then the protein extract consumed will contain 10 milligrams to 5 grams of alkaline phosphatase.

It is recommended that any oral formulation of a less stable alkaline phosphatase (like IALP) is taken orally once a day, while any oral formulation of a more stable alkaline phosphatase (like PLAP) is taken orally once, twice or three times a week. A suitable time to take the oral formulation is just several minutes prior to breakfast in case of a liquid formulation or 1-2 hours prior to breakfast in case of tablet, gel, capsule, or gel capsule formulations. Prior art does not indicate that consumption of an oral alkaline phosphatase formulation should be limited if the subject takes other medication(s) for any disease or disorder.

### EXAMPLES

### Example 1

### Effects of Longer and Shorter Term Treatments with PLAP on the Body Weight of Mice Fed a High Fat Diet (HFD)

Six weeks old female C57BL/6 mice were kept under specified pathogen free (SPF) condition in macrolon cages at 22-24° C. and 50-60% humidity, with lighting regimen of 12/12 h light dark. The animals had free access to tap water and were fed ad libitum either sterilized standard chow diet (˜15% fat, 65% carbohydrate, 20% protein; Charles River VRFI) or a high fat diet (HFD) (˜35% fat, 29% carbohydrate, 30% protein, 6% other components; Special Diets Services #824056; 60% kcal from fat) for 13 weeks. The animals were cared for according to the “Guiding Principles for the Care and Use for Animals” based upon the Helsinki declaration. Treatments with 1.5 mg/mouse of highly purified PLAP were performed either thrice per week from the beginning of week 1 or once per week from the beginning of the 7th week. Administration of 50 μl PLAP solution (made up in physiological saline) was performed subcutaneously. There were 7 animals in each group. The data is expressed as mean±S.D. of seven determinations.

The data, presented in TABLE 2, indicate that compared to mice fed a control chow, feeding a HFD significantly increased body weight over a 13 week period. Longer term treatment with PLAP fully, while the shorter term treatment only partially prevented body weight increase due to HFD. Importantly, at the time when the analysis of various metabolic parameters was performed there was a significant difference in the animals' body weight in the two PLAP treated groups. In the HFD group that received long term PLAP treatment animals were of normal weight, while in the HFD group that received shorter term PLAP treatment the animals were overweight on week 7 and afterward.

TABLE 2
Effects of PLAP treatments on body weight of mice fed HFD.

| | Body weight (g); (n = 7) | | | |
|---|---|---|---|---|
| | | | Group 3 | Group 4 |
| | Group 1 | Group 2 | Fatty diet | Fatty diet |
| Week on | Normal diet | Fatty diet | PLAP-treated | PLAP-treated |
| fatty diet | Untreated | Untreated | 3 x per week | 1 x per week |
| 0 | 22.3 ± 1.06 | 22.7 ± 1.35 | 22.1 ± 0.68 | 22.6 ± 1.20 |
| 1 | 24.0 ± 0.99 | 25.7 ± 1.59 | 25.4 ± 0.61 | 26.0 ± 1.51 |
| 2 | 25.3 ± 0.93 | 26.7 ± 1.98 | 26.0 ± 0.60 | 27.4 ± 1.85 |
| 3 | 26.3 ± 1.07 | 28.2 ± 1.77 | 26.7 ± 0.71 | 28.8 ± 2.29 |
| 4 | 27.1 ± 1.22 | 30.8 ± 1.89 | 28.5 ± 0.54 | 30.2 ± 2.50 |
| 5 | 28.3 ± 1.34 | 32.2 ± 2.09 | 28.2 ± 1.02* | 31.5 ± 2.82 |
| 6 | 28.5 ± 1.61 | 33.9 ± 2.16 | 27.7 ± 1.64* | 32.5 ± 3.00 |
| 7 | 29.1 ± 1.52 | 35.1 ± 2.63 | 28.2 ± 1.66* | 33.1 ± 3.10 |
| 8 | 29.8 ± 1.89 | 36.5 ± 2.97 | 28.3 ± 2.10* | 33.4 ± 3.39 |
| 9 | 30.1 ± 2.30 | 37.6 ± 3.29 | 28.8 ± 2.16* | 33.4 ± 3.49 |
| 10 | 30.2 ± 2.14 | 37.7 ± 3.22 | 28.4 ± 2.10* | 33.5 ± 3.26 |
| 11 | 30.5 ± 2.60 | 38.0 ± 3.28 | 28.8 ± 2.40* | 33.4 ± 3.14 |
| 12 | 30.6 ± 2.31 | 39.2 ± 3.12 | 30.6 ± 2.31* | 33.6 ± 3.05* |
| 13 | 30.0 ± 2.25 | 40.3 ± 3.26 | 28.8 ± 2.15* | 33.3 ± 2.47*^{,}** |

*Significantly (P < 0.01) different from the corresponding mean value in Group 2.
**Significantly (P < 0.05) different from the corresponding mean value in Group 3.

The major conclusion drawn from the data in TABLE 2, in relevance to the effects of PLAP on insulin resistance, hyperinsulinemia, and dyslipidemia, is that the two different PLAP treatment regiments led to significantly different effects on body weight. If the effects of PLAP on insulin resistance, hyperinsulinemia, and dyslipidemia, would strictly relate to its effects on body weight, than the longer term and shorter term treatments with PLAP also should result in significantly different effects on these metabolic events.

The complete prevention of weight gain by the 1.5 mg/mouse dose of PLAP in response to HFD was achieved without affecting food consumption as demonstrated in TABLE 3. One possibility to explain this phenomenon is that PLAP enhanced energy expenditure.

TABLE 3
Effect of PLAP on the consumption of fatty food

| | Food consumption per week (g) | | |
|---|---|---|---|
| | | | HFD |
| | Control diet | HFD | PLAP treatment |
| Week | No treatment | No treatment | 3-times per week |
| 3 | 3.17 | 2.23 | 2.49 |
| 4 | 4.00 | 2.49 | 2.37 |
| 5 | 3.29 | 2.61 | 2.46 |
| 6 | 3.34 | 2.50 | 2.46 |
| 7 | 3.50 | 2.59 | 2.40 |
| 8 | 3.36 | 2.49 | 2.44 |
| 9 | 3.34 | 2.46 | 2.31 |
| 10 | 3.40 | 2.47 | 2.40 |
| 11 | 3.46 | 2.39 | 2.39 |
| 12 | 3.47 | 2.31 | 2.31 |
| 13 | 3.54 | 2.45 | 2.41 |
| Mean | 3.44 ± 0.21 | 2.45 ± 0.10 | 2.40 ± 0.06 |

### Example 2

### Effects of PLAP on Insulin Sensitivity in a Glucose Tolerance Experiment

On the 12th week of the above experiment (described in Example 1), animals were starved for 8 hours followed by the intraperitoneal injection of 1.5 g/kg glucose. In the PLAP 3× group (mice treated with PLAP 3-times a week from week 1), the last treatments were 48 hours prior to glucose injection, while in the PLAP 1× group (mice treated with PLAP once a week from week 7) the last treatments were 6 days prior to glucose injection. Blood samples were collected just before (0 min) as well as 30 min, 60 min and 120 min after glucose injection. Blood samples were taken from the corner of the eyes, and glucose concentrations in whole blood samples were immediately measured with the fast Glucose C test (Wako Chemicals USA Inc., Richmond, Va.). The data, shown in TABLE 4, is expressed as mean±S.D. of measurements from seven mice.

The results, shown in TABLE 4, indicate that by the 90th and 120th min the blood glucose level in both PLAP treated groups were significantly lower than in the untreated HFD fed group. This strongly suggested that using either treatment regimen, PLAP similarly enhanced insulin sensitivity with no discernible, statistically different, differences between the PLAP 3× and PLAP 1× groups. Since the two treatment regimens had statistically different effects on body weight (as demonstrated in TABLE 2), the effects of PLAP on insulin sensitivity in mice, and by extension in humans consuming fatty food, may be only partly due to its effects on body weight; the other part of its effect on insulin sensitivity must be due to a different mechanism. It is also remarkable that although in the PLAP 1× group the last treatment was 6 days prior to the glucose injection, the results on glucose tolerance/insulin sensitivity were still practically the same when the last treatment was 2 days prior to the glucose injection.

TABLE 4
Longer and shorter term treatments with PLAP
similarly enhance insulin sensitivity in HFD fed mice.

| | Blood glucose (mM) | | | |
|---|---|---|---|---|
| Treatment | 0 min | 30 min | 90 min | 120 min |
| Control chow | 4.6 ± 0.5 | 9.3 ± 0.6 | 7.7 ± 0.3 | 5.5 ± 0.4 |
| HFD | 10.8 ± 0.4 | 18.8 ± 1.8 | 15.3 ± 1.9 | 12.5 ± 0.4 |
| HFD + PLAP 3x | 7.5 ± 0.4 | 15.2 ± 1.1 | 11.7 ± 1.1* | 7.8 ± 0.6* |
| HFD + PLAP 1x | 9.8 ± 0.4 | 16.3 ± 0.9 | 12.8 ± 0.7* | 8.2 ± 0.4* |

*Significantly (P < 0.01) different from the corresponding mean value in the HDF group.

### Example 3

### Determination of Serum Insulin Level

First Day
1. Distribute 300 μl of assay buffer to the non-specific binding (NSB) tubes (3-4), 200 μl to reference (Bo) tubes (5-6), and 100 μl to tubes 7 through the end of the assay.
2. Distribute 100 μl of standards and quality controls in duplicate according to the flow chart).
3. Distribute 100 μl of each sample in duplicate.
4. Distribute 100 μl of ultrasensitive mouse antibody (Ultrasensitive Mouse Antibody kit from DRG) to all tubes except total count tubes (1-2) and NSB tubes (3-4).
5. Vortex, cover, and incubate overnight (20-24 hours) at 4° C.

Second Day
6. Distribute 100 μl of ¹²⁵I-Insulin to all tubes.
7. Vortex, cover, and incubate the tubes overnight (20-24 hours) at 4° C.

Third Day
8. Add 1.0 ml of cold (4° C.) precipitating reagent (included in the kit) to all tubes except total count tubes (1-2).
9. Vortex and incubate 20 minutes at 4° C.
10. Centrifuge at 4° C. all tubes (except total count tubes (1-2) for 20 minutes at 2,000-3,000×g.

Conversion of Rpm to x g:

xg=(1.12×10⁻⁵)(r)(rpm)²
r=radial distance in cm (from axis of rotation to the bottom of the tube)
rpm=rotational velocity of the rotor
11. Immediately decant the supernatant of all tubes except total count tubes (1-2), drain tubes for 60 seconds, and blot excess liquid from by inverting the tubes only one time.
12. Count all tubes in a gamma counter for 1 minute. Calculate the amount of mouse insulin in unknown samples using automated data reduction procedures.

### Example 4

### Effects of PLAP on Serum Insulin Level in HFD Fed Mice

At the end of the 13th week of the experiment described in Example 1, the animals were decapitated and blood was collected for serum preparation. The last treatments with PLAP in the PLAP 3× (mice treated with PLAP 3-times a week from week 1) and PLAP 1× (mice treated with PLAP once a week from week 7) groups were performed 48 hours and 6 days prior to decapitation, respectively. Serum insulin levels were determined as described under Example 3. The data, shown in TABLE 5, is expressed as mean±S.D. of measurements from seven mice.

Earlier data in TABLE 4 indicated that long term feeding of mice with HFD resulted in insulin resistance that was reduced in a very similar fashion by both longer and shorter term treatments with PLAP. The data in TABLE 5 show that both the longer term and shorter term treatments with PLAP similarly reduced hyperinsulinemia in HFD fed mice. Since insulin resistance is the cause of hyperinsulinemia, the data in TABLE 4 and TABLE 5 combined further confirm that mice indeed developed insulin resistance.

The effects of long term and shorter term PLAP treatments on HFD-induced hyperinsulinemia were statistically indistinguishable. This means that these effects of PLAP in mice, and by extension in humans consuming fatty diet, may be only partially the consequence of its effects on body weight which were statistically different. That statement also was made in case of PLAP effects on insulin resistance.

A particularly interesting finding, shown in TABLE 5, was that both PLAP treatment regimens reduced the amount of serum insulin below the level seen in animals kept on the control chow. Consequently, PLAP also should promote normalization of blood glucose level in a glucose tolerance test performed with mice on a standard chow diet.

TABLE 5
Longer and shorter treatments with PLAP similarly
reduce serum insulin level in HFD fed mice.

| Treatment | Serum insulin (pmol/L) |
|---|---|
| Control chow | 130.3 ± 36.3 |
| HDF | 250.2 ± 36.0 |
| HDF + PLAP 3x | 71.8 ± 26.5* |
| HDF + PLAP 1x | 97.5 ± 39.2* |

*Significantly (P < 0.001) different from the corresponding mean value in the HDF group.

### Example 5

### Effects of PLAP on Blood Glucose and Insulin Levels in a Glucose Tolerance Test Performed with Mice Fed a Standard Chow Diet

Proteins were purified from Sigma PLAP to 90-100% as described in detail earlier [She, Q.-B., Mukherjee, J. J., Huang, J.-S., Crilly, K. S, and Kiss, Z. (2000) Growth factor-like effects of placental alkaline phosphatase in human fetus and mouse embryo fibroblasts. FEBS Letters, 468, 163-167; Placental alkaline phosphatase to control diabetes; U.S. Pat. No. 7,501,116; issued on Mar. 10, 2009; Inventor: Zoltan Kiss]. In case of PLAP preparation no other protein could be detected with naked eye or densitometry on the SDS-gel after staining with coommassie blue. Albumin, transferrin and α1-antitrypsin were about 90%, 95%, and ˜100% pure, respectively, as determined by densitometry.

C57BL/6 female mice weighing 22-25 g, specified pathogen free (SPF) hygienic category from Charles River VRF₁ were used. The animals were kept in macrolon cages at 22-24° C. and 50-60% humidity with lightning regimen of 12/12 hours light/dark. They had free access to tap water and were fed with a sterilized standard diet (Charles River VRF₁, autoclavable). The animals were cared for according to the “Guiding Principles for the Care and Use of Animals” based upon the Helsinki Declaration and the experiments were approved by the local ethical committee.

Animals were subcutaneously administered 1.2 mg/mouse or 0.3 mg/mouse of proteins, as indicated in TABLE 6, 24 hours prior to the intraperitoneal administration of 3 g/kg of glucose. Animals were also starved for 12 hours prior to glucose administration. Blood samples were taken few minutes before glucose administration (0 min) and then 30 min, 60 min, and 120 min after glucose administration from the corner of eye (canthus). Blood glucose was measured with the Fast Glucose C-test. The data, shown in TABLE 6, is expressed as mean±S.D. of measurements from five mice.

In line with the ability of PLAP to reduce serum insulin level even in mice kept on control chow diet (TABLE 5), in these animals PLAP also significantly reduced the blood glucose level in a glucose tolerance test. PLAP was almost as effective at the dose of 0.3 mg per mouse as at the dose of 1.2 mg per mouse. Overall these results strongly indicate that even in normoglycemic subjects PLAP can enhance insulin sensitivity, so that in the presence of PLAP less insulin is needed for the same effect. Furthermore, the results also indicated that, in relatively short term experiments, the optimum dose of PLAP to reduce blood glucose in mice is between 0.3 and 1.2 mg PLAP per mouse.

Although no other protein was detectable in the final PLAP preparation used for this experiment, it was useful to compare the effects of purified PLAP to the major contaminating proteins that were present in the starting commercial PLAP preparation [Placental alkaline phosphatase to control diabetes; U.S. Pat. No. 7,501,116; issued on Mar. 10, 2009; Inventor: Zoltan Kiss]. It is clear from the data presented in TABLE 6 that only PLAP had significant and consistent effects on blood glucose level.

TABLE 6
Reduction of blood glucose by PLAP in glucose tolerance test
performed with mice fed a control chow diet: Comparison of
PLAP effects to that of other proteins present in the
commercial PLAP preparation.

| | Blood glucose (mM) | | | |
|---|---|---|---|---|
| Protein (mg/mouse) | 0 min | 30 min | 60 min | 120 min |
| None | 4.8 ± 0.4 | 11.3 ± 0.6 | 9.8 ± 0.6 | 6.4 ± 0.5 |
| PLAP, 1.2 | 4.9 ± 0.6 | 7.7 ± 0.8 | 5.0 ± 0.5 | 4.8 ± 0.3 |
| PLAP, 0.3 | 5.0 ± 0.4 | 8.3 ± 0.6 | 5.7 ± 0.3 | 5.5 ± 0.3 |
| Albumin, 1.2 | 5.1 ± 0.3 | 10.4 ± 0.9 | 7.5 ± 0.5 | 6.8 ± 0.3 |
| Transferrin, 1.2 | 4.6 ± 0.4 | 11.5 ± 0.4 | 9.6 ± 0.4 | 7.5 ± 0.4 |
| α1-antitrypsin, 1.2 | 5.4 ± 0.5 | 11.5 ± 0.5 | 10.1 ± 0.8 | 9.0 ± 0.6 |

In a similar experiment described in Example 5, three groups of 7 week old (23-26 g) female C57BL/6 mice kept on control chow diet were used for the determination of serum insulin level using the method described under Example 3. All animals were starved overnight prior to intraperitoneal injection of 3 g per kg glucose. In the first group (0 time, no glucose) mice were only starved but received no glucose or PLAP treatment. In this group, blood was taken about 35-40 min prior to taking blood from the other two groups. In the second group (30 min+glucose), starved mice received only glucose and blood was drawn 30 min after glucose injection. In the third group (30 min+glucose+PLAP), mice were subcutaneously administered 1.2 mg of PLAP per mouse 24 hours prior to glucose administration to starved mice followed by taking the blood 30 min after glucose injection. Serum insulin was determined with the “Ultrasensitive Mouse Antibody” kit as indicated under Example 3. The data, shown in TABLE 7, is expressed as mean±S.D. of measurements from five mice.

Taken together the data in TABLE 6 and TABLE 7 one can conclude that PALP can simultaneously reduce blood glucose level (TABLE 6) and serum insulin level (TABLE 7) in mice exposed to excess glucose. The most reasonable explanation that can be offered is that, as demonstrated earlier, PLAP enhances insulin sensitivity of peripheral tissues resulting in faster removal of glucose from the blood. Since the extent of insulin release is related to blood glucose level, faster clearance of blood glucose will result in reduced insulin secretion.

The major implication of data in TABLE 7 is that PLAP is suitable to reduce hyperinsulinemia caused by high glycemic carbohydrate which in turn should reduce both postprandial increase in serum FFA and insulin resistance. Fast food, consumed by many in modern age, is composed of high fat and high-glycemic index carbohydrates. The data presented so far indicate that PLAP is suitable to prevent or reduce insulin resistance and hyperinsulinemia in response to both high fat and high-glycemic index carbohydrates.

TABLE 7
PLAP reduces the increase in serum insulin level
in response to a large glucose load.

| Treatment | Serum insulin (pmol/L) |
|---|---|
| 0 time, no glucose | 109 ± 15 |
| 30 min, + glucose | 238 ± 31 |
| 30 min, + glucose + PLAP | 149 ± 26* |

*Significantly (P < 0.01) different from the mean value in the “only glucose” group.

### Example 6

### Effects of PLAP on Serum Lipids in Mice Fed HFD

Dyslipidemia, characterized by increased serum and tissue levels of long chain saturated FFAs and triglycerides as well as an increase in the ratio of LDL-C to HDL-C, is the most significant contributor to insulin resistance and hyperinsulinemia as discussed in the “Background” section. In particular, increased consumption of fatty food leading to high serum levels of FFAs is a critical factor in the development of insulin resistance, associated hyperinsulinemia, and other components of dyslipidemia. Thus, reduction of elevated serum FFAs by PLAP would serve as a strong predictor for reduced insulin resistance and associated hyperinsulinemia. Reduction of dyslipidemia would either prevent or reduce insulin resistance and hyperinsulinemia depending on how early the alkaline phosphatase treatment regimen is started; the earlier the treatment starts, the greater the chance that insulin resistance and hyperinsulinemia can be prevented. The two experiments presented under this example serve to determine if longer and shorter term treatments with PLAP can reduce dyslipidemia.

In the first experiment, at the end of the 13th week of treatments as described in Example 1, the animals were decapitated and blood was collected for serum preparation. Diagon kits were used for the determination of triglyceride (TG) and total cholesterol (TC) using Olympus 640 equipment, as described in the manufacturer's instructions. The last treatments with PLAP in the PLAP 3× (mice treated with PLAP 3-times a week from week 1) and PLAP 1× (mice treated with PLAP once a week from week 7) groups were performed 48 hours and 6 days prior to decapitation, respectively. The data, shown in TABLE 8, is expressed as mean±S.D. of measurements from seven mice.

The results clearly indicate that both longer and shorter treatments with PLAP prevented increases in the serum content of both triglyceride and total cholesterol. The differences in the effects of PLAP after longer term or shorter term treatments were not statistically different. In contrast, longer and shorter term treatments with PLAP caused statistically different effects on body weight (TABLE 2). Thus, the ability of PLAP to reduce serum lipids in mice on HDF, and by extension in humans consuming fatty diet, could only partly relate to its effects on body weight. The other conclusion that can be drawn from the experiment presented in TABLE 8 is that by preventing the increase in the triglyceride and total cholesterol components of dyslipidemia, PLAP is a suitable agent to prevent insulin resistance and hyperinsulinemia.

TABLE 8
Both long term and short term PLAP treatments prevent HFD-induced
increase in serum triglyceride (TG) and total cholesterol (TC).

| | TG | TC |
|---|---|---|
| Treatment | (mmol/L) | (mmol/L) |
| Control chow | 0.72 ± 0.07 | 3.1 ± 0.28 |
| HFD | 2.15 ± 0.53 | 6.4 ± 0.71 |
| HFD + PLAP 3x | 0.65 ± 0.05* | 3.8 ± 0.50* |
| HFD + PLAP1x | 0.87 ± 0.27* | 3.7 ± 0.34* |

*Significantly (P < 0.001) different from the corresponding mean value in the HDF group.

The second experiment was performed as the first one, except that the mice kept on HFD were treated (subcutaneously) for 13 weeks, twice a week, with 0.5 mg of PLAP per mouse. The last treatment with PLAP was performed 48 hours prior to collecting the blood from the eye corner after fasting for 12 hours. Serum lipids were determined from the serum within 4 hours. An automatic Hitachi 747 automatic chemical analyzer was used to determine triglycerides (GPO-PAP reagents; Roche Diagnostic GmbH, Mannheim, Germany), total cholesterol (SICDIA LT-CHO reagents; Eiken Chemical Industries, Tokyo, Japan), HDL-C (Cholestest-HDL reagents; Daiichi Chemicals, Tokyo, Japan), and LDL-C (Cholestest-LDL reagents Daiichi Chemicals, Tokyo, Japan). Fatty acid was determined by using the NEFA C kit (Wako Chemicals). The data, shown in TABLE 9, is expressed as mean±S.D. of measurements from seven mice.

The first important observation was that at the dose of 0.5 mg per mouse, PLAP was practically as effective as the 1.5 mg per dose in reducing serum triglyceride while somewhat less effective in reducing total cholesterol (compare data in TABLE 8 and TABLE 9). However, PLAP increased the ratio of HDL-C (good cholesterol) to LDL-C (bad cholesterol) from 2.63 to 4.60. Although in C57BL/6 mice the basal level of HDL-C to LDL-C is higher than in humans, the data nevertheless indicate that HFD reduced the HDL-C/LDL-C ratio while PLAP treatment increased it. This implies that in dyslipidemic humans as well PLAP treatment may result in a more favorable ratio between HDL-C and LDL-C.

The second most important observation was that PLAP also significantly reduced the serum level of FFAs. Since all these HFD-induced changes in lipids, and particularly increased serum level of FFAs, significantly contribute to insulin resistance and hyperinsulinemia, the observed effects of PLAP (as demonstrated in TABLE 9) further confirm the earlier conclusion that by reducing HFD-induced dyslipidemia in mice, and by implication in humans, PLAP can prevent or reduce insulin resistance and associated hyperinsulinemia.

TABLE 9
PLAP strongly reduces the components of dyslipidemia in mice
fed HFD.

| Lipid | Control chow | HFD | HFD + PLAP |
|---|---|---|---|
| TG, (mmol/L) | 0.41 ± 0.12 | 0.90 ± 0.21 | 0.48 ± 0.09* |
| TC, (mmol/L) | 2.25 ± 0.41 | 5.83 ± 0.90 | 3.88 ± 0.70* |
| HDL-C, (mmol/L) | 1.26 ± 0.26 | 3.29 ± 0.43 | 2.35 ± 0.29* |
| LDL-C, (mmol/L) | 0.38 ± 0.16 | 1.16 ± 0.24 | 0.51 ± 0.12* |
| FFA, (mmol/L) | 0.63 ± 0.22 | 1.31 ± 0.19 | 0.87 ± 0.15* |
| HDL-C/LDL-C | 3.31 | 2.83 | 4.60 |

*Significantly (P < 0.01) different from the corresponding mean value in the HDF group.

### Example 7

### Short Term Effects of Orally Administered PLAP on Serum FFAs

Six to seven weeks old male C57BL/6 mice were fed either the standard chow diet or the HFD for 3 days. The animals (5 in each group) had free access to either water without any further addition or water containing 1 mg/ml of purified PLAP. Under this condition PLAP (which is fully soluble in water) remains stable. The mice did not appear to develop any aversion to drinking the protein containing water. In agreement with data presented in TABLE 2, after 3 days on HFD there was no statistically different change in body weight compared to mice on control chow diet. Thus this animal model corresponds to a normal weight model consuming HFD for a limited time without gaining weight. Similarly, PLAP treatment for 3 days also had no statistically different effect on body weight. After 3 days and a 6 hour long starvation period the FFA content of serum was determined by using the NEFA C kit (Wako Chemicals). The data, shown in TABLE 10, is expressed as mean±S.D. of 5 determinations.

In mice fed a control chow, PLAP did not change serum FFA content. HFD nearly doubled the amount of serum FFA, and this was significantly reduced by orally administered PLAP. This result indicates that orally administered PLAP is capable of reducing serum FFA in mice on high fat diet, and by implication in humans consuming fatty food. In view of the strong involvement of elevated serum FFAs in insulin resistance and hyperinsulinemia as well as other aspects of dyslipidemia (enhanced levels of triglyceride, cholesterol and ceramide), oral consumption of PLAP is a suitable administration route to prevent or at least reduce dyslipidemia as well as insulin resistance and hyperinsulinemia.

The site of the action of orally administered PLAP is presently unknown. However, similar to lactoferrin [Takeuchi, T., Kitagawa, H. and Harada, E. (2004) Evidence of lactoferrin transportation into blood circulation from intestine via lymphatic pathway in adult rats. Exp. Physiol. 80, 263-270] it may be transported into the blood circulation from the intestine via the lymphatic pathway and there may help the clearance of FFAs.

TABLE 10
Orally administered PLAP reduces serum FFA
in mice fed HFD for 3 days.

Free fatty acid (mmol/L)

| Diet | No PLAP | +PLAP |
|---|---|---|
| Control chow | 0.54 ± 0.12 | 0.49 ± 0.10 |
| HFD | 0.96 ± 0.07 | 0.67 ± 0.13* |

*Significantly (P < 0.01) different from the corresponding mean value in the HDF group.

1. A method of preventing or reducing one or more interrelated metabolic disorders selected from the group consisting essentially of dyslipidemia, insulin resistance, and hyperinsulinemia, comprising the step of administering a therapeutically effective amount of an alkaline phosphatase preparation to prevent or reduce the one or more metabolic disorders and as a consequence prevent or reduce development of related diseases and disorders in a human or another mammal.

2. The method of claim 1 wherein the alkaline phosphatase is placental alkaline phosphatase, intestinal alkaline phosphatase, tissue-nonspecific alkaline phosphatase, or germ cell alkaline phosphatase.

3. The method of claim 2 wherein the alkaline phosphatase is homogenous placental alkaline phosphatase.

4. The method of claim 1 wherein the alkaline phosphatase is extracted and highly purified from a corresponding human or animal tissue, or from suitable cells and organisms.

5. The method of claim 1 wherein the alkaline phosphatase is a recombinant protein.

6. The method of claim 1 wherein the alkaline phosphatase is chemically synthesized.

7. The method of claim 1 wherein the alkaline phosphatase is an active derivative of placental alkaline phosphatase.

8. The method of claim 7 wherein the active derivative is produced by genetic modification of placental alkaline phosphatase resulting in a recombinant product.

9. The method of claim 7 wherein the active derivative is chemically synthesized, or derived from placental alkaline phosphatase by a proteolytic process.

10. The method of claim 1 wherein the alkaline phosphatase is a recombinant alkaline phosphatase produced from plant seeds or plant leaves as a plant-derived partially purified protein extract.

11. The method of claim 10 wherein alkaline phosphatase makes up 0.1% to 50% of the plant-derived protein extract.

12. The method of claim 1 wherein the step of administering is performed by infusion, with a minipump ensuring controlled release, or by intravenous, intraarterial, intraportal, subcutaneous, intraperitoneal, intramuscular, or intradermal injection of a preparation comprising:
a. a physiologically acceptable carrier; and
b. highly purified alkaline phosphatase dissolved or dispersed in the carrier.

13. The method of claim 12 wherein the therapeutically effective amount is in the range of 0.01 grams to 2.5 grams per square meter of calculated body surface of the mammal.

14. The method of claim 12 wherein the therapeutically effective amount is in the range of 0.1 grams to 1.0 grams per square meter of calculated body surface of the mammal.

15. The method of claim 1 wherein the step of administering is performed orally in a suitable formulation.

16. The method of claim 15 wherein the formulation is a liquid, tablet, gel, capsule, gel-capsule, or a semisolid mixture containing suitable food components.

17. The method of claim 15 wherein the alkaline phosphatase is highly purified.

18. The method of claim 15 wherein the alkaline phosphatase is a recombinant alkaline phosphatase produced from plant seeds or plant leaves as a plant-derived partially purified protein extract.

19. The method of claim 15 wherein any formulation for one-time oral consumption by a subject weighing 100 kg contains 1 mg to 500 mg of alkaline phosphatase.

20. The method of claim 15 wherein any formulation for one-time oral consumption by a subject weighing 100 kg contains 20 mg to 200 mg of alkaline phosphatase.

21. The method of claim 1 wherein the alkaline phosphatase is placental alkaline phosphatase and it is administered once, twice, or three-times per week.

22. The method of claim 1 wherein the related diseases and disorders to be prevented or reduced include type 2 diabetes, epithelial cancers, myopia, acne, acanthosis nigricans, polycystic ovary syndrome, cutaneous papillomas, hypertension, renal dysfunction, microvascular and macrovascular diseases, atherosclerosis, nonalcoholic fat liver, dyslipidemia, sleep-disordered breathing, atherosclerosis, coronary artery (heart) disease, cardiovascular disease, heart failure, accelerated aging, stroke, and neurological diseases such as Alzheimer.

23. The method of claim 22 wherein the alkaline phosphatase is used together with medications prescribed for the related diseases and disorders.

24. The method of claim 23 wherein the medication is an inhibitor of 3-hydroxy-3-methylglutaryl coenzyme A reductase (statin) used with or without niacin.

25. The method of claim 23 wherein the alkaline phosphatase is used together with metformin.

26. A therapeutically effective amount of an alkaline phosphatase preparation for the treatment of metabolic disorders selected from a group consisting essentially of dyslipidemia, insulin resistance, and hyperinsulinemia.
